(19) United States
(12) Patent Application Publication
(10) Pub. No.: US 2019/0060087 A1
(43) Pub. Date: Feb. 28, 2019

(54) YELLOW HAMMER CUP IMPACTOR

(71) Applicant: K. David Moore, Vestavia Hills, AL (US)

(72) Inventor: K. David Moore, Vestavia Hills, AL (US)

(21) Appl. No.: 15/682,645

(22) Filed: Aug. 22, 2017

(51) Int. Cl. A61F 2/46

(52) U.S. Cl. CPC A61F 2/4609; A61F 2002/30001

(57) ABSTRACT

A cup impactor has a shaft having a distal end, a proximate end, and an intermediate section between the distal end and the proximate end. A cup adapter for a cup may be disposed at the distal end, the cup adapter adapted to detachably retain the cup during a hip replacement operation. A modular plate adapter for a modular plate may be disposed at the proximate end, the modular plate adapter adapted to detachably retain the modular plate during the hip replacement operation, the modular plate adapted to at least one of an impactor, a mallet, a pneumatic hammer, or a robotic arm. An attachment point for an array of transmitters may be disposed at the intermediate section, the array of transmitters coupled to a receiver, the array of transmitters transmitting location and orientation data of the cup impactor to the receiver.

### BACKGROUND

### 1. Field

The application relates to a cup impactor for inserting for use during orthopedic surgery.

### 2. Description of the Related Art

Components may be inserted into a surgical site, such as a bone, during orthopedic surgery. A cup impactor provides a way to hold the component while it is projected into the surgical site.

### SUMMARY

In one aspect, a cup impactor has a shaft having a distal end, a proximate end, and an intermediate section between the distal end and the proximate end; a cup adapter for a cup may be disposed at the distal end, the cup adapter adapted to detachably retain the cup during a hip replacement operation; a modular plate adapter for a modular plate may be disposed at the proximate end, the modular plate adapter adapted to detachably retain the modular plate during the hip replacement operation, the modular plate adapted to at least one of an impactor, a mallet, a gas or electric-powered hammer, or a robotic arm; and an attachment point for an array of transmitters may be disposed at the intermediate section, the array of transmitters coupled to a receiver, the array of transmitters transmitting location and orientation data of the cup impactor to the receiver.

The above and other features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated herein and form part of the specification, illustrate various embodiments of the present invention and, together with the description, further serve to explain the principles and to enable a person skilled in the pertinent art to make and use the invention. In the drawings, like reference numbers indicate identical or functionally similar elements. A more complete appreciation and many of the attendant advantages thereof will be readily obtained as the same becomes better understood by reference to the following detailed description when considered in connection with the accompanying drawings, wherein:

FIG. 1 shows a cup impactor 100 according to an embodiment;

FIG. 2 shows a cup impactor 200 according to an embodiment;

FIG. 3 shows a cup impactor 300 according to an embodiment;

FIG. 4 shows a cup impactor 400 according to an embodiment;

FIG. 5 shows a cup impactor 500 according to an embodiment;

FIG. 6 shows a cup impactor 600 according to an embodiment;

FIG. 7 shows a cup impactor 700 according to an embodiment;

FIG. 8 shows a cup impactor 800 according to an embodiment; and

FIG. 9 shows a cup impactor 900 according to an embodiment.

### DETAILED DESCRIPTION OF THE PREFERRED EMBODIMENTS

In several embodiments, the Yellowhammer Cup Impactor provides a more accurate and safer means of placing the acetabular component during total hip replacement while diminishing surgeon fatigue.

Currently, the acetabular component is inserted manually. The cup is temporarily fixed, usually by a screw mechanism to an impaction arm which the surgeon strikes with a mallet. This impaction can require a good deal of force.

Accurate placement of the acetabular component in total hip replacement is a crucial element in avoiding complications during and after total hip replacement. Intra-operative x-rays and computer assisted and robotic acetabular preparations are methods that have been utilized to augment surgical expertise in reproducibly accurate acetabular component placement. The Yellowhammer Cup Impactor would allow real-time assessment of cup placement via an array attached to the impactor providing information to a computer or robot guided system.

Fracture of the pelvis during acetabular component placement is a known potential complication of hip replacement surgery that can lead to a more extensive surgery, increase risk of blood loss, infection and loosening. One of the causes of acetabular fracture could be from the non-uniform forces generated in manual impaction by the surgeon. A gas or electrically powered impaction device either free-standing or as part of a robot system would allow more uniform force to be applied during cup impaction decreasing the risk of fracture and decrease surgeon fatigue which could, in turn, lead to other errors.

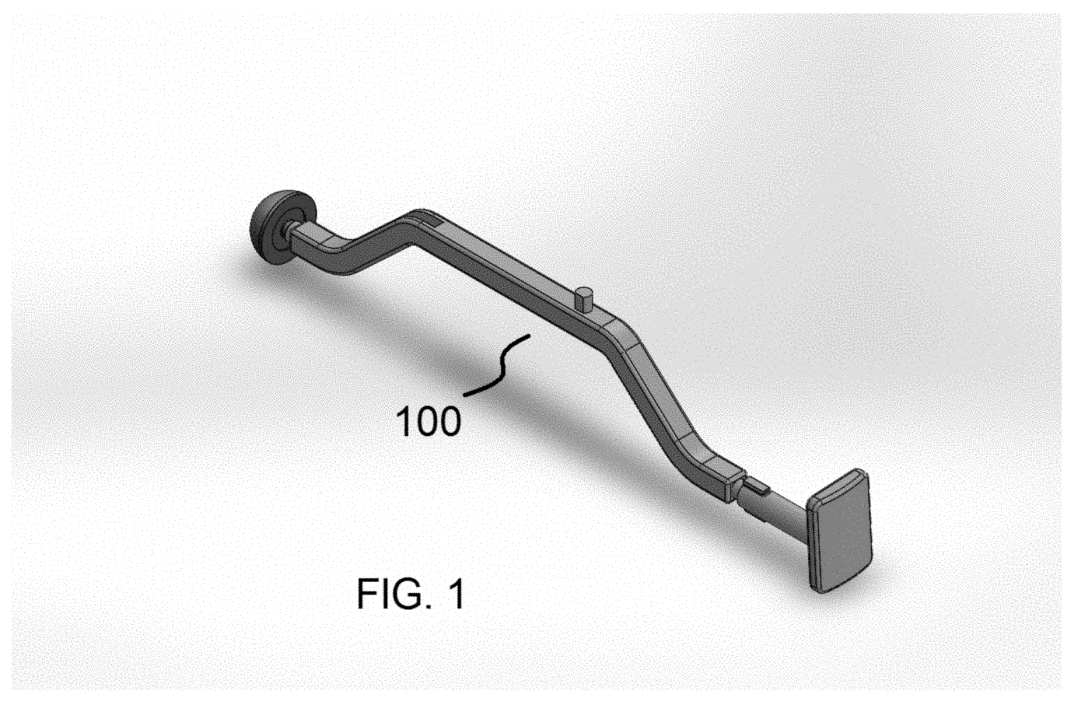

In FIG. 1 is shown a cup impactor 100 according to an embodiment.

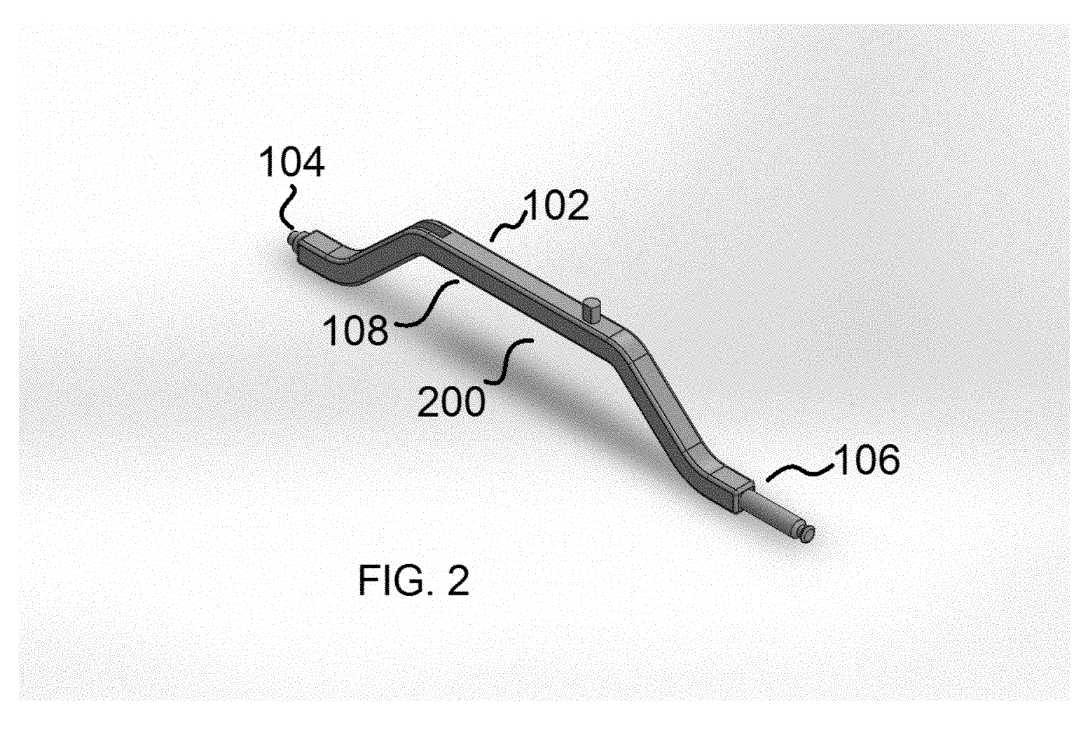

In FIG. 2 is shown a cup impactor 200 according to an embodiment. As shown in FIG. 2, the cup impactor 200 includes a shaft 102 having a distal end 104 and a proximate end 106. An intermediate section 108 extends between the distal end 104 and the proximate end 106 of the cup impactor 200. In one embodiment, the intermediate section 108 is offset from a centerline 124 of the distal end 104 and the proximate end 106.

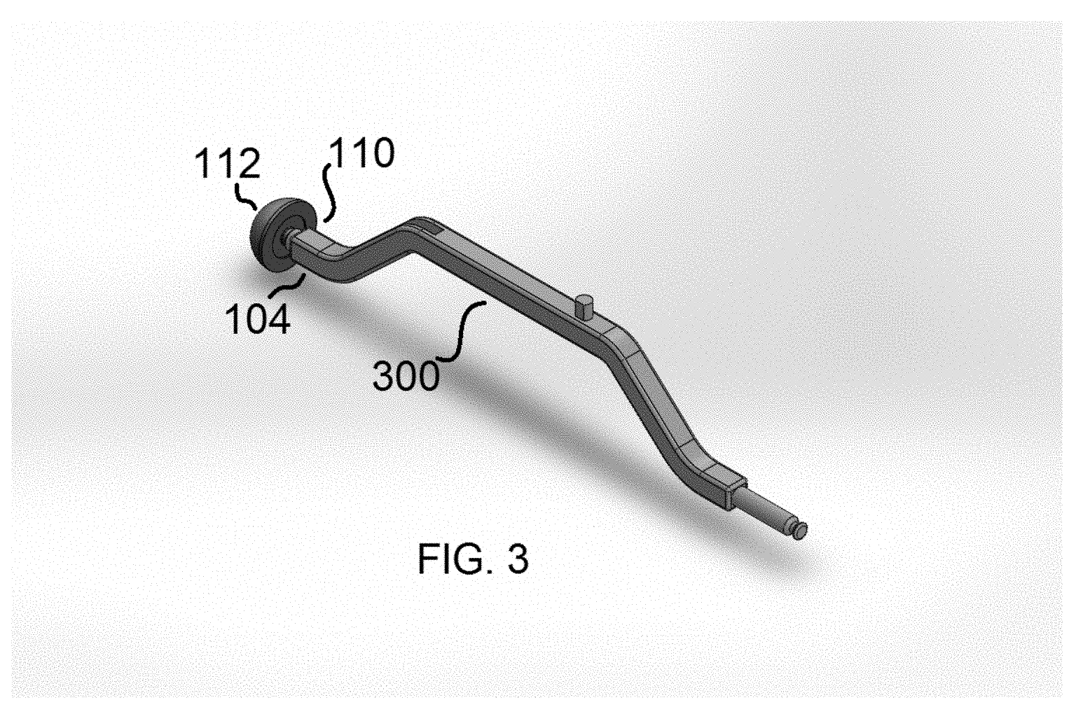

In FIG. 3 is shown a cup impactor 300 according to an embodiment. As shown in FIG. 3, a cup adapter 110 for a cup 112 is disposed at the distal end 104 of the cup impactor 300. The cup adapter 110 is adapted to detachably retain the cup 112 during a hip replacement operation.

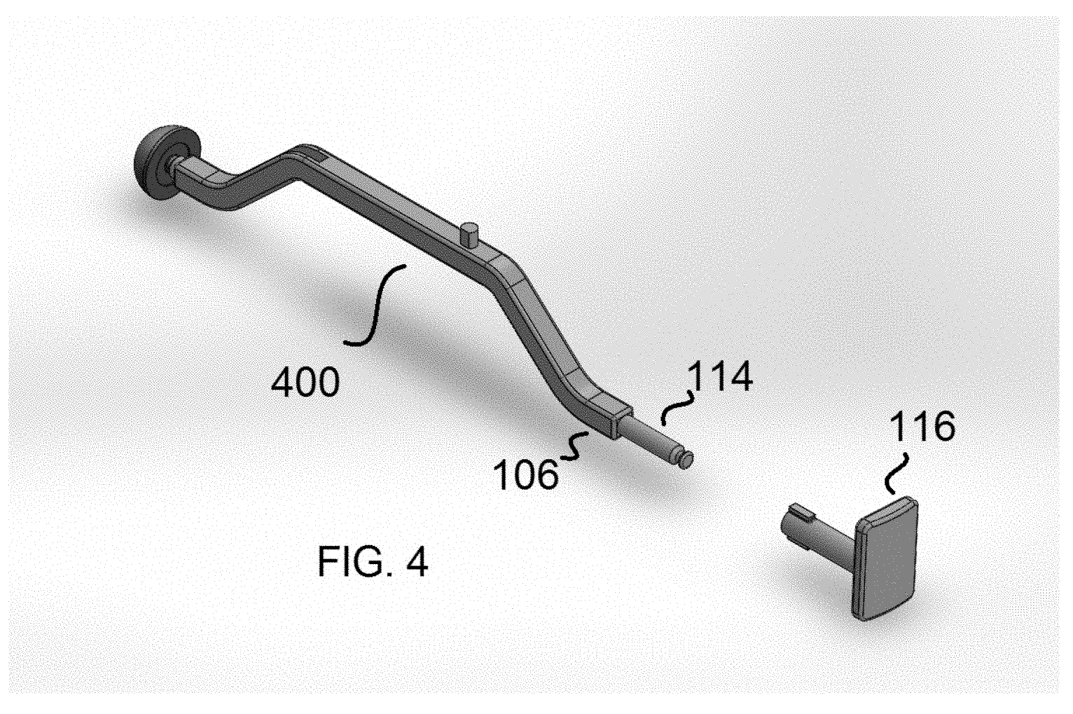

In FIG. 4 is shown a cup impactor 400 according to an embodiment. As shown in FIG. 4, a modular plate adapter 114 for a modular plate 116 is disposed at the proximate end 106 of the cup impactor 400. The modular plate adapter 114 is adapted to detachably retain the modular plate 116 during the hip replacement operation.

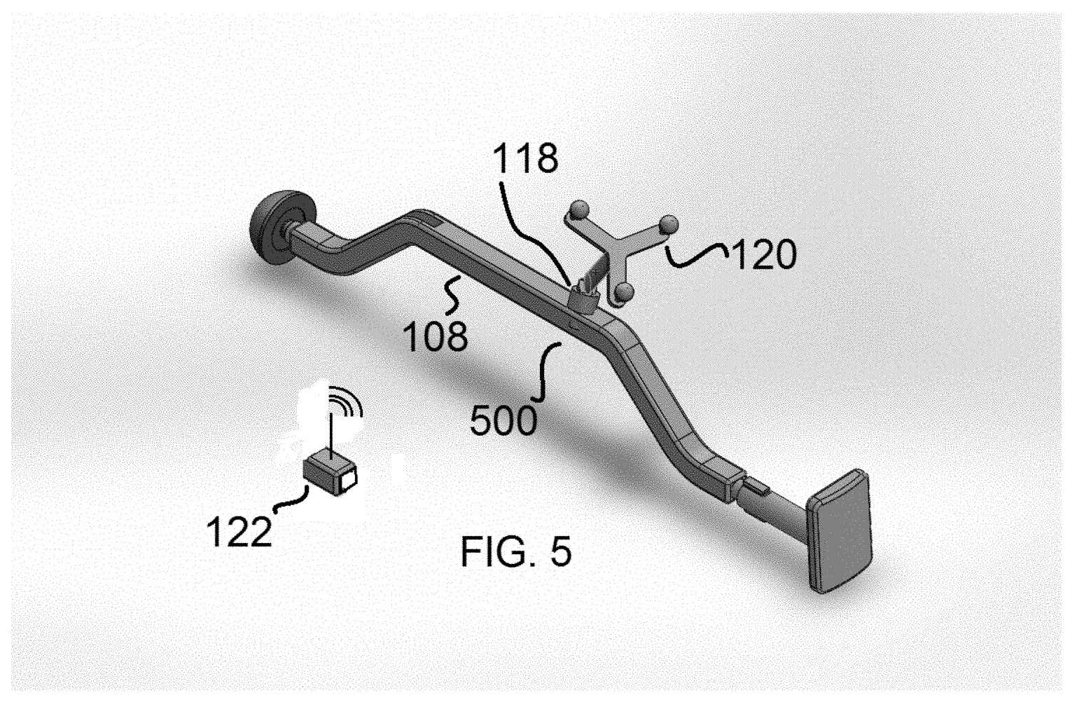

In FIG. 5 is shown a cup impactor 500 according to an embodiment. As shown in FIG. 5, an attachment point 118 for an array of transmitters 120 is disposed at the intermediate section 108 of the cup impactor 500. The array of transmitters 120 is coupled to a receiver 122. The array of transmitters 120 transmits location and orientation data of the cup impactor 100 to the receiver 122.

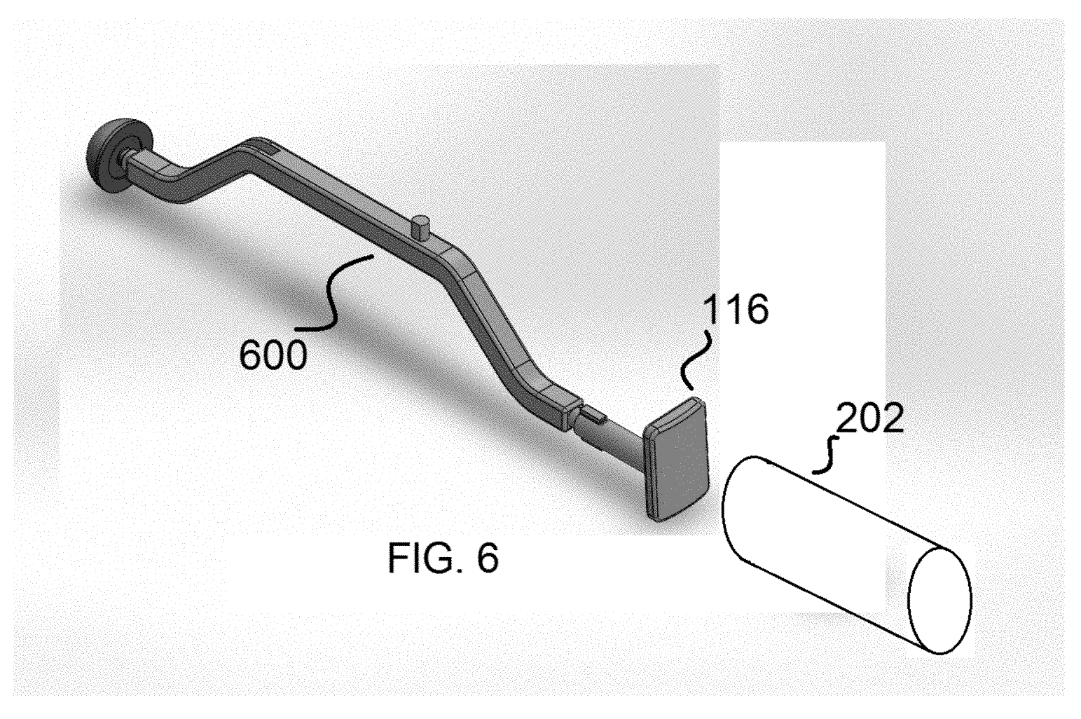

In FIG. 6 is shown a cup impactor 600 according to an embodiment. As shown in FIG. 6, the modular plate 116 of the cup impactor 600 is adapted to an impactor 202.

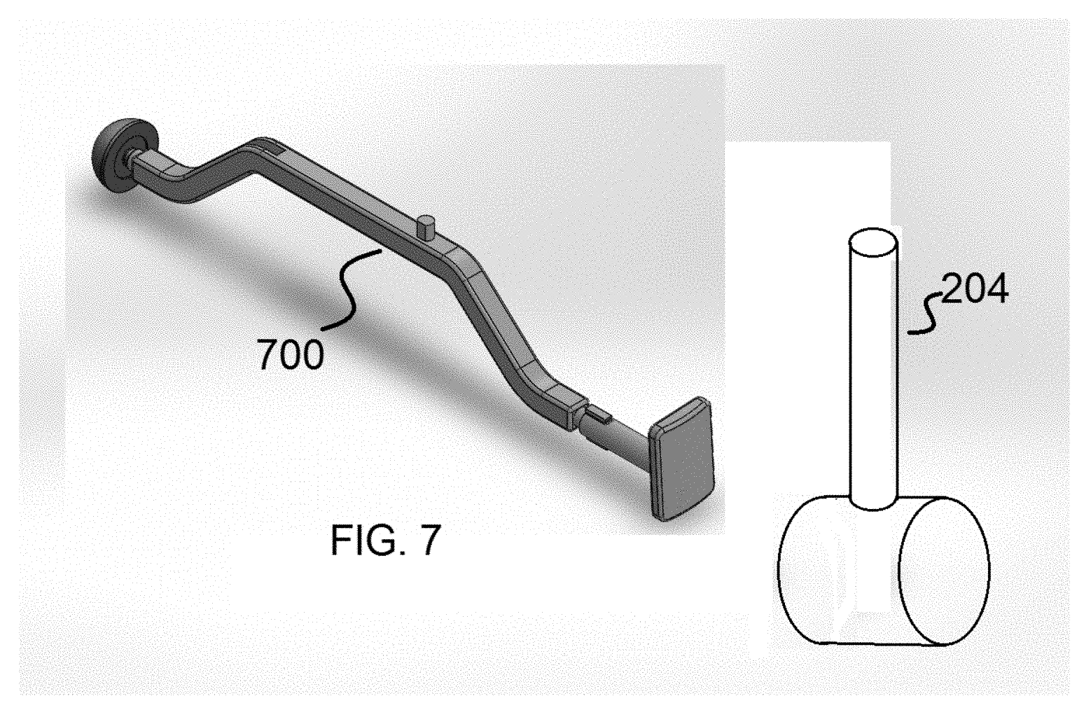

In FIG. 7 is shown a cup impactor 700 according to an embodiment. As shown in FIG. 7, the impactor is a mallet 204.

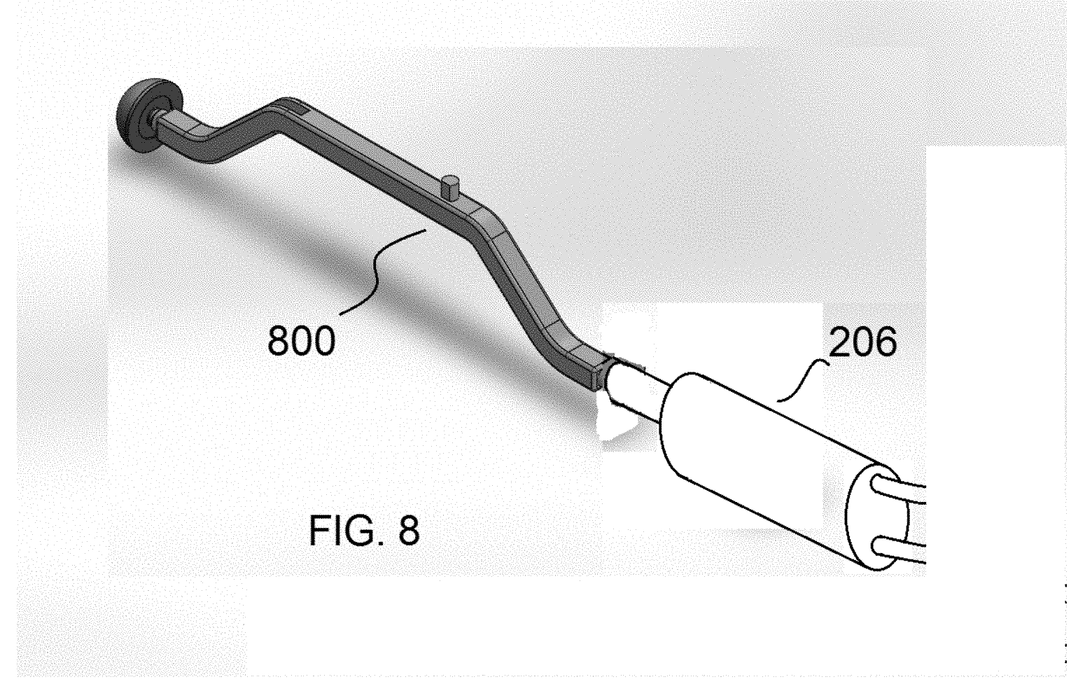

In FIG. 8 is shown a cup impactor 800 according to an embodiment. As shown in FIG. 8, the impactor is a gas or electric-powered hammer 206. In one embodiment, the receiver 122 modulates a force or an oscillation frequency of the gas or electric-powered hammer 206 in response to the location and the orientation data. In one embodiment, the force is 0.8 N to 1 N. In one embodiment, the oscillation frequency is 70 Hz.

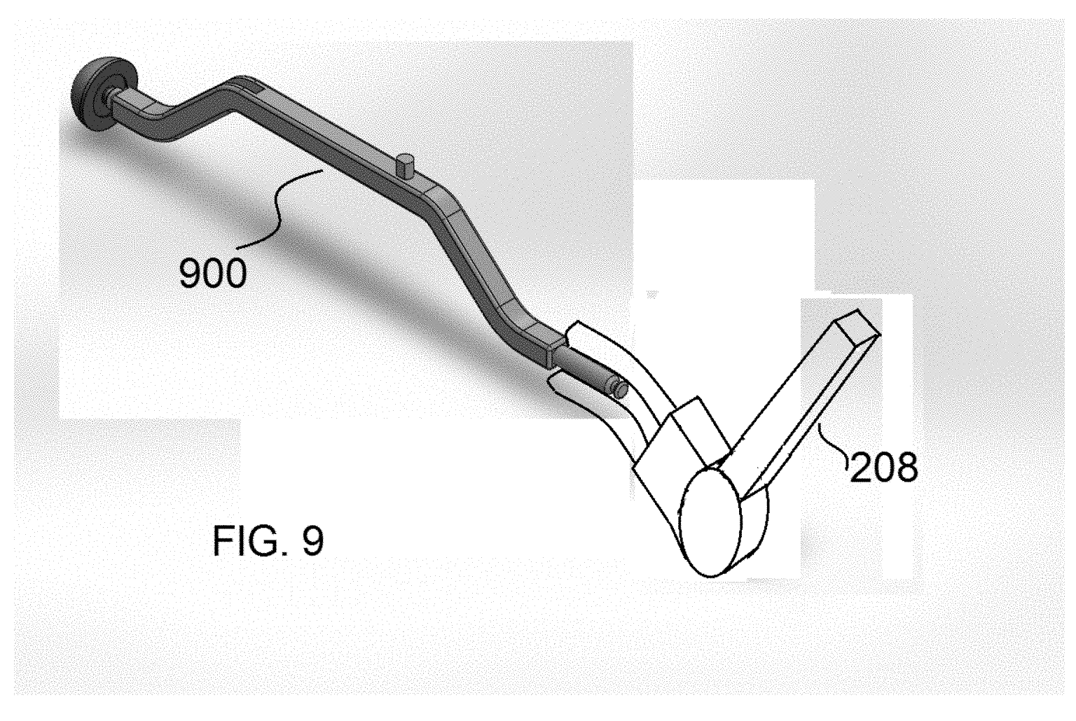

In FIG. 9 is shown a cup impactor 900 according to an embodiment. As shown in FIG. 9, the impactor is a robotic arm 208. In one embodiment, the receiver 122 guides the robotic arm 208 in response to the location and the orientation data.

The foregoing has described the principles, embodiments, and modes of operation of the present invention. However, the invention should not be construed as being limited to the particular embodiments described above, as they should be regarded as being illustrative and not restrictive. It should be appreciated that variations may be made in those embodiments by those skilled in the art without departing from the scope of the present invention.

While a preferred embodiment of the present invention has been described above, it should be understood that it has been presented by way of example only, and not limitation. Thus, the breadth and scope of the present invention should not be limited by the above described exemplary embodiment.

Obviously, numerous modifications and variations of the present invention are possible in light of the above teachings. It is therefore to be understood that the invention may be practiced otherwise than as specifically described herein.

What is claimed is:

1. A cup impactor comprising:
a shaft having a distal end, a proximate end, and an intermediate section between the distal end and the proximate end;
a cup adapter for a cup disposed at the distal end, the cup adapter adapted to detachably retain the cup during a hip replacement operation;
a modular plate adapter for a modular plate disposed at the proximate end, the modular plate adapter adapted to detachably retain the modular plate during the hip replacement operation, the modular plate adapted to at least one of an impactor, a mallet, a pneumatic hammer, or a robotic arm; and
an attachment point for an array of transmitters disposed at the intermediate section, the array of transmitters coupled to a receiver, the array of transmitters transmitting location and orientation data of the cup impactor to the receiver.

2. The cup impactor of claim 1, where the impactor is the pneumatic hammer, and the receiver modulates a force or an oscillation frequency of the pneumatic hammer in response to the location and the orientation data.

3. The cup impactor of claim 1, where the impactor is the robotic arm, and the receiver guides the robotic arm in response to the location and the orientation data.

4. The cup impactor of claim 1, where the intermediate section is offset from a centerline of the distal end and the proximate end.
